(19) United States
(12) Patent Application Publication
(10) Pub. No.: US 2003/0045836 A1
(43) Pub. Date: Mar. 6, 2003

(54) Redirectional catheter apparatus

(76) Inventor: Stanley Batiste, Las Vegas, NV (US)

Correspondence Address: HENDERSON & STURM LLP, 1213 MIDLAND BUILDING, 206 SIXTH AVENUE, DES MOINES, IA, 50309-4076, US

(21) Appl. No.: 09/944,446

(22) Filed: Aug. 31, 2001

(51) Int. Cl. A61M 5/178; A61M 25/00

(52) U.S. Cl. 604/164.13; 604/264

(57) ABSTRACT

A redirectional catheter apparatus 10 for inserting a conventional catheter 80 into a desired vascular access 201 wherein the redirectional catheter apparatus 10 includes a redirectional catheter member 20 having an elongated hollow stem 21 which includes a slightly tapered distal end 24, an enlarged proximal end 23, and a deflection flap 26 disposed proximate to, but spaced from, the distal end of the hollow stem 21 wherein the deflection flap 26 forms an aperture 25 in the sidewall 27 of the hollow stem 21 for directing a secondary guide wire 101 into the desired vascular access 201 so that the secondary guide wire 101 will eventually guide the conventional catheter 80 into the desired vascular access 201 and the method employed to accomplish that result.

### BACKGROUND OF THE INVENTION

1. Field of the Invention

The present invention relates to the field of surgical implements in general and in particular to a method and apparatus for properly positioning a catheter into a desired vascular access artery.

2. Description of Related Art

As can be seen by reference to the following U.S. Pat. Nos. 5,807,324; 5,993,424; 6,045,530; 6,110,164; and 6,159,187, the prior art is replete with myriad and diverse surgical implements and methods of employing the same.

While all of the aforementioned prior art constructions are more than adequate for the basic purpose and function for which they have been specifically designed, they are uniformly deficient with respect to their failure to provide a simple, efficient, and practical method and apparatus for properly positioning and installing a conventional catheter into a desired vascular access. Accessing a desired vessel in a patient's vascular system, such as the common femoral artery (CFA), can only be made within a very small region with increased risk of complication if the artery is punctured too low or too high. Once accessed, the CFA quickly bifurcates into the deep and superficial femoral arteries. The superficial femoral artery provides blood flow to the lower leg and is generally the intended vessel for vascular access. Once the CFA is punctured, a guide wire is advanced through the needle. In a high percentage of cases, the guide wire will advance into the deep femoral artery. Until now, redirection is very difficult as the needle must be pulled back and, without removal from the vessel, the wire must be redirected. During this procedure, the vessel walls and tissues cannot be seen, only the radio-opaque wire and needle are visualized. If the needle is pulled back too far and access is lost, there is increased risk for the patient due to bleeding and/or vessel injury as a second puncture must be performed.

As a consequence of the foregoing situation, there has existed a lorigstanding need among surgeons for a new and improved redirectional catheter apparatus and its method of use that will simplify the insertion of a catheter into a desired artery with minimum vessel damage to the patient; and the provision of such an arrangement is the stated objective of the present invention.

### BRIEF SUMMARY OF THE INVENTION

Briefly stated, the redirectional catheter apparatus that forms the basis of the present invention comprises in general a catheter member having an elongated stem provided with a slightly tapered distal end, a proximal end having an enlarged mouth, and an aperture disposed proximate to, but spaced from, the distal end wherein the side aperture is formed by a deflection flap that extends into the interior of the hollow stem.

As will be explained in greater detail further on in the specification, the crux of this invention resides in the deflection flap and the aperture which are both created in the preferred embodiment of the invention by severing a portion of the sidewalls of the hollow stem.

In addition, the positioning of the side aperture and the deflection flap at a location proximate to but spaced from the distal end of the redirectional catheter member is crucial to the method of using the apparatus of this invention to install a conventional catheter in a desired vascular access.

The preliminary positioning of the redirectional catheter apparatus is accomplished by threading the proximal end of a primary guide wire previously inserted into the vascular system through the distal end of the redirectional catheter apparatus. Then the primary guide wire is removed so that a secondary guide wire can be inserted through the proximal end of the redirectional catheter member thereby engaging the deflection flap to direct the secondary guide wire through the side aperture and into a desired vascular access.

At this juncture, the redirectional catheter apparatus is removed and the secondary guide wire guides a conventional catheter into the desired vascular access.

### BRIEF DESCRIPTION OF THE SEVERAL VIEWS OF THE DRAWINGS

These and other attributes of the invention will become more clear upon a thorough study of the following description of the best mode for carrying out the invention, particularly when reviewed in conjunction with the drawings, wherein:

FIG. 1 is a perspective view of the redirectional apparatus and a primary guide wire;

FIG. 2 is an isolated detail view of the distal end of the redirectional apparatus;

FIG. 3 is an isolated detail view of the first position of the guide wire relative to the redirectional apparatus;

FIG. 4 is an exploded perspective view of the guide wire and the redirectional apparatus;

FIG. 5 is an isolated detail view of the position of a secondary guide wire relative to the redirectional apparatus;

FIG. 6 is an isolated detail view of the secondary guide wire being redirected by the redirectional apparatus;

FIG. 7 shows the insertion of the guide wire into the common femoral artery via a needle;

FIG. 8 shows the position of the primary guide wire when the needle is removed;

FIG. 9 shows the insertion of the redirectional apparatus over the primary guide wire;

FIG. 10 shows the redirectional apparatus after the primary guide wire has been removed;

FIG. 11 shows the redirection of a secondary guide wire by the redirectional apparatus to position the secondary guide wire within the superficial femoral artery prior to guiding a catheter therein;

FIG. 12 shows the redirectional apparatus removed from a surrounding relationship with the secondary guide wire;

FIG. 13 shows a conventional catheter being guided into the vascular system by the secondary guide wire; and,

FIG. 14 shows the secondary guide wire removed from the conventional catheter to complete the installation of the conventional catheter into the proper artery.

### DETAILED DESCRIPTION OF THE INVENTION

As can be seen by reference to the drawings, and in particular to FIG. 1, the redirectional catheter apparatus that forms the basis of the present invention is designated generally by the reference number 10. The apparatus 10 comprises a redirectional catheter member 20 having a generally conventional catheter configuration which includes an elongated hollow stem 21 having an enlarged mouth 22 formed on its proximal end 23 and a slightly tapered distal end 24.

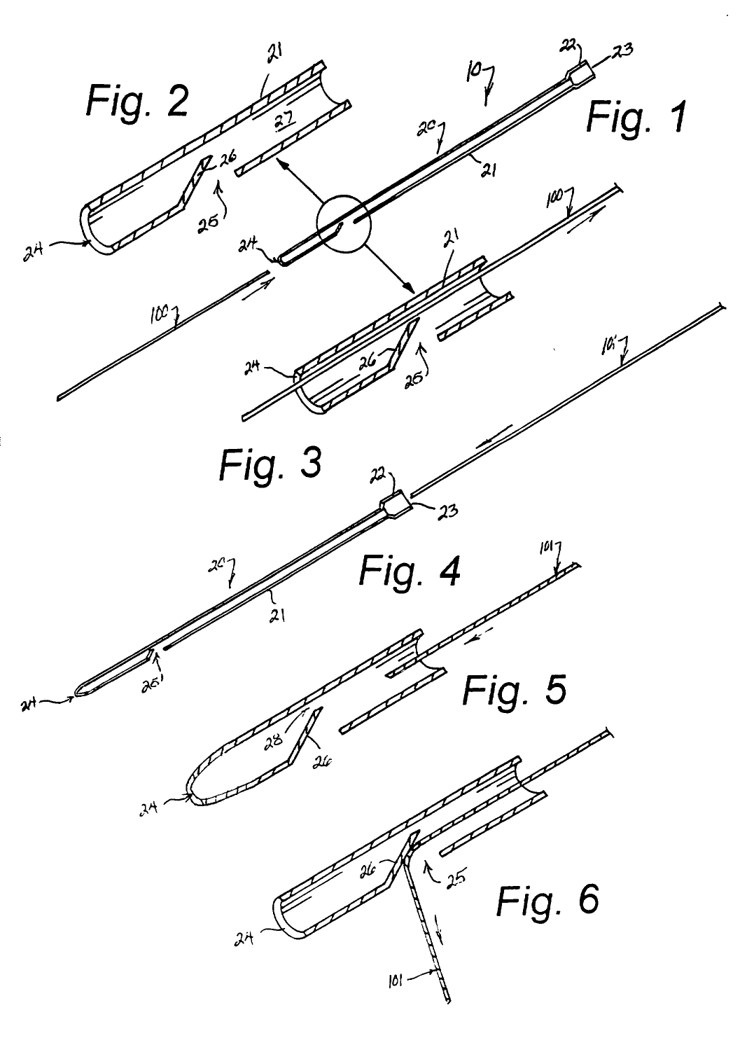

In addition, as shown in FIGS. 2 and 3, an enlarged aperture 25 is formed proximate to, but spaced from, the distal end 24 by a deflection flap 26 that is created by severing a portion of the sidewall 27 of the hollow stem 21 and forcing the severed edges of the flap 26 into the interior of the hollow stem 21 to partially obstruct the longitudinal passageway of the hollow stem 21 of the catheter member 20 for reasons that will be explained presently.

Prior to embarking on a detailed description of the preferred method of using the apparatus 10 to redirect the installation of a conventional catheter (not shown) into a patient's vascular system, it should be noted by reference to FIGS. 3 through 6, that the deflector flap 26 is disposed in such a manner within the hollow stem 21 of the redirectional catheter member 20 so as to easily accept the passage of the proximal end of a primary guide wire 100 through the distal end 24 of the redirectional catheter member 20 wherein the primary guide wire 100 will slide up the forwardly angled rear surface of the deflection flap 26 to pass through the restricted opening 28 in the interior of the hollow stem 21 of the catheter member 20.

However, when the distal end of a secondary guide wire 101 is inserted into the proximal end of the redirectional catheter member 20, the distal end of the secondary guide wire 101 will be blocked by the forwardly angled front surface of the deflection flap 26 to redirect the distal end of the secondary guide wire 101 out through the side aperture 25 in the hollow stem 21 of the catheter member 20.

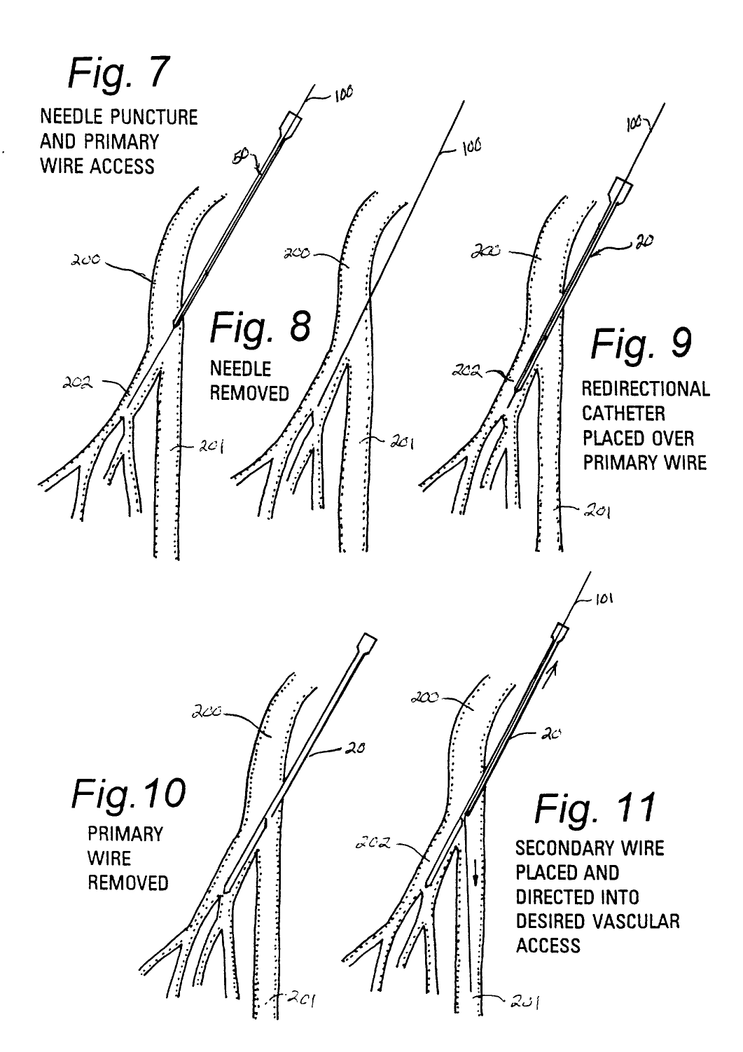

Turning now to FIGS. 7 through 11, it can be seen that the apparatus 10 is employed in the following method to insure that a conventional catheter will be guided into the desired vascular entry.

To begin with, as depicted in FIG. 7, a hollow needle 50 is inserted into the common femoral artery 200 above the juncture of the superficial 201 and branched deep 202 femoral arteries and the primary guide wire 100 is threaded through the needle 50.

At this point, the needle 60 is withdrawn as shown in FIG. 8, and the proximal end of the primary guide wire 100 is inserted into the distal end 24 of the redirectional catheter member 20 as shown in FIGS. 1 and 3, so that the distal end 24 of the catheter member 20 will enter the juncture of the superficial 201 deep 202 femoral arteries as depicted in FIG. 9.

Once the redirectional catheter apparatus 10 has been properly positioned, the primary guide wire 100 is removed as shown in FIG. 10, and the distal end of a secondary guide wire 101 is then inserted into the proximal end 23 of the catheter member 20 as shown in FIG. 3, wherein the distal end of the secondary guide wire 101 will encounter the deflection flap 26 and be forced downwardly through the side aperture 25 in the side of the hollow stem 21 of the catheter member 20 as shown in FIGS. 4, 5, and 11 to place the distal end of the secondary guide wire 101 into the superficial femoral artery 201.

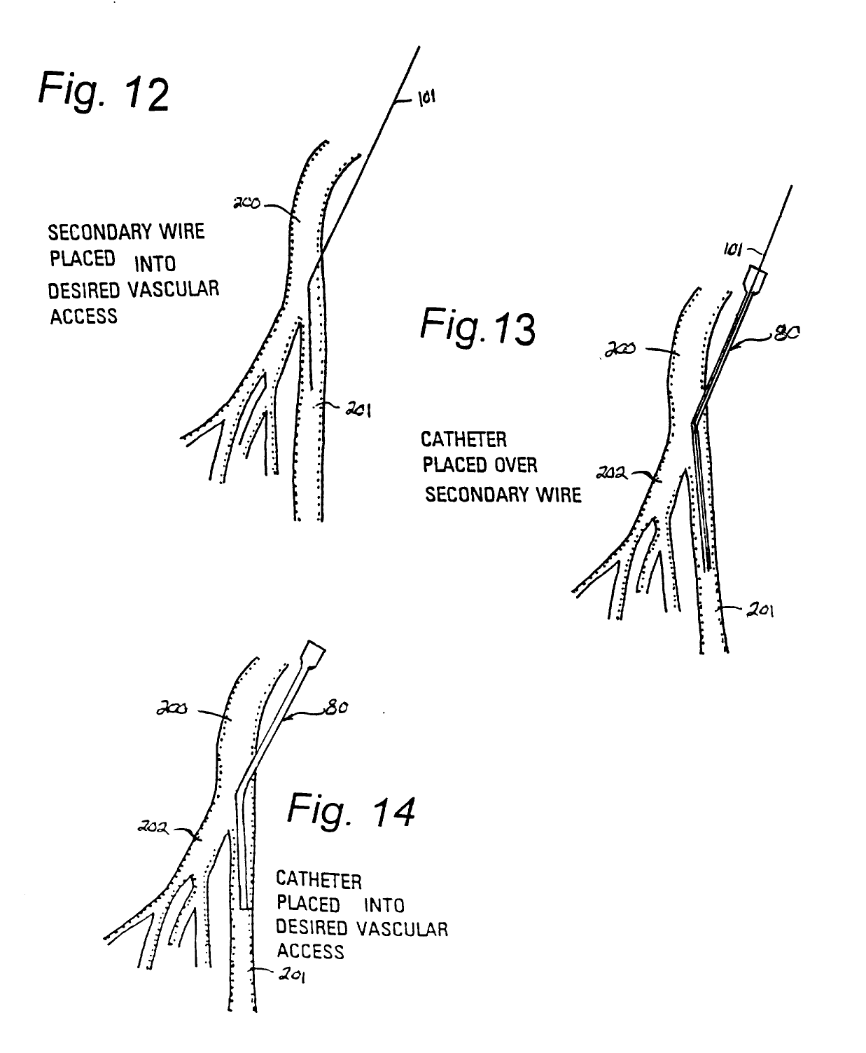

At this juncture, the redirectional catheter apparatus 10 is withdrawn from a surrounding relationship with the secondary guide wire 101 which remains in the superficial femoral artery 201 as depicted in FIG. 12; and, the secondary guide wire 101 is used to guide a conventional catheter 80 into the superficial femoral artery as shown in FIG. 13.

Once the conventional catheter 80 is properly seated within the superficial femoral artery 201, the secondary guide wire 101 is removed to complete the installation of the conventional catheter 80 in the patient's vascular system.

Although only an exemplary embodiment of the invention has been described in detail above, those skilled in the art will readily appreciate that many modifications are possible without materially departing from the novel teachings and advantages of this invention. Accordingly, all such modifications are intended to be included within the scope of this invention as defined in the following claims.

In the claims, means-plus function clauses are intended to cover the structures described herein as performing the recited function and not only structural equivalents, but also equivalent structures.

Having thereby described the subject matter of the present invention, it should be apparent that many substitutions, modifications, and variations of the invention are possible in light of the above teachings. It is therefore to be understood that the invention as taught and described herein is only to be limited to the extent of the breadth and scope of the appended claims.

I claim:

1. A redirectional catheter apparatus for use in combination with a hollow needle, a primary guide wire having a distal end and a proximal end, a secondary guide wire having a distal end and a proximal end and a conventional catheter having a distal end and a proximal end for properly positioning the conventional catheter in a desired vascular access in a patient; wherein, the apparatus comprises
a redirectional catheter member including an elongated hollow stem having a distal end and a proximal end provided with an enlarged mouth wherein the hollow stem is provided with an aperture which is disposed proximate to, but spaced from, the distal end of the hollow stem and is dimensioned to receive the secondary guide wire and wherein the distal and proximal ends of the hollow stem are dimensioned to receive the primary guide wire.

2. The apparatus as in claim 1, further comprising:
first means for permitting the passage of the primary guide wire along the entire longitudinal length of the hollow stem.

3. The apparatus as in claim 2, further comprising:
second means adapted to contact the distal end of the secondary guide wire for deflecting the distal end of the secondary guide wire through said aperture.

4. The apparatus as in claim 1, further comprising
a deflection flap operatively associated with said aperture and adapted to permit the passage of the primary guide wire over the entire length of the hollow stem when the primary guide wire is inserted through the distal end of the hollow stem and adapted to block the passage of a secondary guide wire from the proximal to the distal end of the hollow stem when the distal end of the secondary guide wire is inserted through the proximal end of the hollow stem.

5. The apparatus as in claim 4; wherein, the deflection flap is further adapted to divert the distal end of the secondary guide wire through said aperture.

6. The apparatus as in claim 5; wherein, the deflection flap is formed integrally with said hollow stem.

7. The apparatus as in claim 6; wherein, the hollow stem has sidewalls and the deflection flap is defined by severed portions of the sidewalls of the hollow stem.

8. The apparatus as in claim 4; wherein, the deflection flap is angled forwardly in the direction of the proximal end of the hollow stem.

9. The apparatus as in claim 3; further comprising a forwardly angled deflection flap disposed adjacent said aperture and having a front surface and a rear surface.

10. The apparatus as in claim 9; wherein, the first means comprises the rear surface of the deflection flap.

11. The apparatus as in claim 10; wherein the second means comprises the front surface of the deflection flap.

12. The apparatus as in claim 1; wherein, the distal end of the redirectional catheter member has a tapered configuration.

13. A redirectional catheter apparatus in combination with a hollow needle, a primary guide wire having a distal end and a proximal end, a secondary guide wire having a distal end and a proximal end and a conventional catheter having a distal end and a proximal end for properly positioning the conventional catheter in a desired vascular access in a patient; wherein, the apparatus comprises
a redirectional catheter member including an elongated hollow stem having a slightly tapered distal end and a proximal end provided with an enlarged mouth wherein the hollow stem is provided with an aperture which is disposed proximate to, but spaced from, the distal end of the hollow stem and is dimensioned to receive the secondary guide wire and wherein the distal and proximal ends of the hollow stem are dimensioned to receive the primary guide wire.

14. The apparatus as in claim 12; a deflection flap operatively associated with said aperture and adapted to permit the passage of the primary guide wire over the entire length of the hollow stem when the primary guide wire is inserted through the distal end of the hollow stem and adapted to block the passage of a secondary guide wire from the proximal to the distal end of the hollow stem when the distal end of the secondary guide wire is inserted through the proximal end of the hollow stem.

15. The apparatus as in claim 13; wherein, the deflection flap is further adapted to divert the distal end of the secondary guide wire through said aperture.

16. The apparatus as in claim 14; wherein, the deflection flap is formed integrally with said hollow stem.

17. The apparatus as in claim 15; wherein, the hollow stem has sidewalls and the deflection flap is defined by severed portions of the sidewalls of the hollow stem.

18. The apparatus as in claim 13; wherein, the deflection flap is angled forwardly in the direction of the proximal end of the hollow stem.

19. The apparatus as in claim 12; wherein, said primary guide wire is dimensioned to pass through said hollow needle and to pass through the entire length of the redirectional catheter member when the proximal end of the primary guide wire is inserted through the distal end of the redirectional catheter member.

20. The apparatus as in claim 18; wherein, the secondary guide wire is adapted to pass through the aperture in the hollow stem of the redirectional catheter member and to pass completely through the conventional catheter.

21. A method of installing a conventional catheter into a desired vascular access using a hollow needle, a primary guide wire, a secondary guide wire and a redirectional catheter apparatus having a hollow stem provided with a distal end, a proximal end and a deflection flap defining an aperture disposed proximate to, but spaced from, the distal end of the hollow stem including the steps of:
a) introducing the hollow needle into a desired artery;
b) passing the primary guide wire through the hollow needle so that the distal end of the primary guide wire extends into an arterial junction;
c) removing the hollow needle;
d) passing the proximal end of the primary guide wire through the distal end of the redirectional catheter member so that the aperture in the hollow stem of the redirectional catheter member is disposed adjacent a desired vascular access;
e) withdrawing the primary guide wire from the redirectional catheter member;
f) inserting the distal end of the secondary guide wire through the proximal end of the redirectional catheter member;
g) engaging the distal end of the secondary guide wire with said deflection flap to direct the distal end of the secondary guide wire into said desired vascular access;
h) withdrawing the redirectional catheter member from the secondary guide wire;
i) inserting the proximal end of the secondary guide wire into the distal end of a conventional catheter;
j) employing the secondary guide wire to guide the conventional catheter into the desired vascular access; and,
k) withdrawing the secondary guide wire from the conventional catheter.
